(19) United States
(12) Patent Application Publication
(10) Pub. No.: US 2005/0084454 A1
(43) Pub. Date: Apr. 21, 2005

(54) Compositions and methods for cleaning nasal cavities

(75) Inventor: Charles A. Fust, Englewood, FL (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: SinoFresh HealthCare, Inc.

(21) Appl. No.: 10/963,700

(22) Filed: Oct. 13, 2004

Related U.S. Application Data
(60) Provisional application No. 60/511,040, filed on Oct. 14, 2003.

(51) Int. Cl. A61L 9/04; A61K 31/44; A61K 31/40; A61K 31/14

(52) U.S. Cl. 424/45; 514/352; 514/426; 514/642

(57) ABSTRACT

Compositions and methods for cleaning nasal cavities are provided. Representative compositions include an active agent having a quaternary ammonium salt and a pharmaceutically acceptable carrier suitable for intranasal delivery. The compositions are formulated to reduce odors without triggering non-allergic rhinitis when delivered intranasally. The methods include cleaning the nasal cavities of a mammal by nasally delivering to the mammal a composition including a cleaning agent and a pharmaceutically acceptable carrier.

### CROSS-REFERENCE TO RELATED APPLICATIONS

The present application claims the benefit of co-pending U.S. provisional patent application Ser. No. 60/511,040, filed Oct. 13, 2003, entitled, “Compositions And Methods For Cleaning Nasal Cavities,” which is hereby incorporated by reference in its entirety.

### BACKGROUND

1. Technical Field

The present disclosure is generally related to compositions and methods for reducing respiratory odors, and more particularly, to compositions and methods for cleaning nasal cavities.

2. Related Technology

Respiratory odors such as halitosis or “bad breath” typically occur as a result of poor dental hygiene and may also be indicative of lung or sinus infection, uremia, or cirrhosis of the liver. Halitosis is typically controlled through the use of oral treatments including mouthwashes, breath mints, candy, chewing gum, and brushing and flossing teeth. These conventional treatments generally incorporate an odor-masking agent that has a stronger scent than the odors due to halitosis. Because these conventional treatments for halitosis focus on oral causes of halitosis, they do not address nasal cavity contributions to halitosis that can occur, for example, in the absence of adequate cleaning of the nasal passages.

Many problems have been associated with inadequate cleansing of the nasal and sinus cavities. For example, smokers of cigars and cigarettes often have a particular form of halitosis known as “smoker's breath.” Further, other individuals suffering from problems associated with the nasal passages and sinus cavities become addicted to over-the-counter medicated nasal and sinus sprays. Active ingredients in these sprays alter normal body functions to achieve short-term results. Prolonged use of medicated sprays frequently results in user dependency. In many cases this occurs long after the original symptoms for which the sprays were intended have run their course.

Additionally, a particular type of halitosis originates in an area deep within the throat, near the extreme rear of the tongue, where the sphenoid sinus drains into the throat. Bacteria buildup in this area produces sulfurous gases, which results in accumulative fetid breath. Small plastic devices, described as tongue scrapers, are a common method people often use in an attempt to dislodge the bacterial build up as they attempt to combat this type of fetid breath; however, many of the users of these devices report vexatious retching with foreign objects placed so deeply within the throat.

Conventional cleaning of the nasal passages is performed using a saline mist or solution that generally relies on the physical pressures exerted by the saline on the nasal surfaces to clean the nasal surfaces. As a result, the use of saline irrigation to the clean the nasal or sinus passages can be uncomfortable and typically ineffective at comfortably dislodging or removing fluid, mucous, or debris from the nose. Compositions other than saline can be used to irrigate the nasal passages; however, the use of solutions containing scented compounds to mask respiratory odors may induce non-allegeric rhinitis in some individuals. Other individuals having problems with nasal and sinus passages are those recovering from post-operative medical care that are frequently required to visit an ear, nose and throat specialist (ENT) for sinus and nasal irrigation procedures. In these procedures, solutions are forced, under pressure, into the patient's affected areas for cleansing.

Individuals living in certain parts of the country such as farm belts and urban suburbs frequently must contend with very high pollen concentrations contaminating the atmosphere. This environmental condition often results in distressed breathing among a great number of people because their airways become heavily taxed when the pollen, which resembles a very fine dust, becomes entrapped in their nose and sinuses. The pollen also results in sneezing and watery eyes. Particulate matter can become attached to surfaces in the nose requiring a high degree of force to remove them.

Certain geographic areas, such as large, industrialized cities have very poor air quality due to high concentrations of airborne pollution caused by industrial and engine emissions, as well as common mold spores, dust and other hazardous particulates. The pollutants can lodge or even clog the nasal passages and sinus, possibly leading to infection.

A further possible problem with nasal and sinus passages is associated with the “common cold,” a minor respiratory illnesses caused by a variety of different respiratory viruses. Symptoms of nasal discharge, nasal congestion and sneezing are usually present from the first day and usually progress to maximum severity by the second or third day. The cost of treatment with over-the-counter medications and outpatient clinics is estimated at more than $6.5 billion annually, all related to the relief of symptoms. The symptomatic treatment available often contains one or more antihistamines, decongestants, antitussives and expectorants, as well as other types of pharmaceutical actives. For individuals with certain medical conditions such as heart disease, hypertension, diabetes, or thyroid disorder, these active ingredients could pose a risk.

In a wide range of respiratory infections, increased nasal secretion of mucus greatly reduces the efficiency of the sense of smell and taste, and also frequently creates the sensation of unusual taste and smell. This is sometimes reported as a “metallic effect.” A heavy cold has a similar effect of damping odor sensitivity by the over-production of mucus and the swelling of the nasal membranes. A specific infection of the olfactory membrane, known as rhinitis, may result in an altered or total, temporary loss of smell. The same effect may stem from a number of allergies, all of which are associated with nasal congestion.

Some individuals who are required to communicate extensively, such as sales people and teachers, frequently experience excessive dryness of the mouth and nose. This dryness often results in unusual bad breath, as well as a soreness and discomfort of the throat.

Further, an altered sensitivity to odors is commonly encountered during pregnancy. Substances previously pleasant smelling may become repugnant to expectant mothers. The exact reason for this condition is not known, but the body's changed hormonal state exerts an influence on the olfactory membrane, frequently causing it to swell. Additionally, nasal blockage is commonly associated with pregnancy.

Nasal cleaning can be particularly troublesome for individuals having sensitive nasal cavity surfaces. Using nasal cleaning solutions containing scented substances, perfumes, flavoring agents or other odor-masking or odor-blocking agents often triggers physical reactions in individuals having, for example, vasomotor rhinitis. Therefore, currently existing nasal cleaning solutions are not suitable for use by these individuals.

In today's market place, one can find healthcare products related to every conceivable hygienic purpose except for the nose and sinuses. For existing oral care products, what are often marketed as “new and improved formulas” are actually nothing more than old concepts. Mouthwash rinses are used to flush the mouth with bacteria killing agents and, in most cases, with additives of various types designed to freshen the mouth and mask odors. Bacterial elimination is normally accomplished by the use of alcohol and/or various dilutions of saline solution. The use of alcohol in these products often causes the intrusive effect experienced by many persons as a burning sensation. Saline solutions are very effective bacteria-killing agents and do not cause the burning sensation; however, the taste is not well received by most consumers. While somewhat effective in some cases, mouthwashes and other oral means of controlling breath odors may not be completely effective in solving many of the problems noted above.

Thus, a heretofore unaddressed need exists in the industry to address the aforementioned deficiencies and inadequacies.

### SUMMARY

Aspects of the present disclosure are generally directed to compositions and methods for cleaning nasal cavities, including nasal sinuses. Representative compositions include an active agent having a quaternary ammonium salt in combination with a pharmaceutically acceptable carrier suitable for intranasal delivery. The compositions are formulated to reduce odors without triggering non-allergic rhinitis when delivered intranasally.

Other aspects of the disclosure provide compositions having an active agent consisting essentially of a quaternary ammonium salt in an amount effective to reduce odors without triggering non-allergic rhinitis when delivered intranasally, in combination with a pharmaceutically acceptable carrier suitable for intranasal delivery. Suitable quaternary ammonium salts have the following formula: 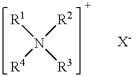
wherein N has a valency of 5; R¹, R², R³, and R⁴ are the same or different and are independently chosen from H, an alkyl group, an alkoxy group, a cycloalkyl group, a heterocycloalkyl group, an aryl group, a heteroaryl group, an acyl group, or a thioacyl group; and X is an anion, preferably a halogen.

Suitable ammonium salts include monoalkyltrimethyl ammonium salts, monoalkyldimethylbenzyl ammonium salts; dialkyldimethyl ammonium salts; and heterocyclic ammonium salts. Pyridinium quaternary salts, particularly substituted pyridinium quaternary salts such and bisquaternary ammonium salts are also useful in the disclosed compositions.

The compositions of the present disclosure can include additional agents, for example, mucolytic agents, surfactants, anti-microbials, desensitizing agents, anti-histamines, and anti-inflammatory agents, or combinations thereof.

Other systems, methods, features, and advantages of the disclosed compositions and methods will be or become apparent to one with skill in the art upon examination of the following drawings and detailed description. It is intended that all such additional systems, methods, features, and advantages be included within this description, be within the scope of the present disclosure, and be protected by the accompanying claims.

### DETAILED DESCRIPTION

Embodiments of the present disclosure include compositions and methods for cleaning or refreshing nasal cavities, in particular for cleaning and refreshing nasal cavities without triggering non-allergic rhinitis. One exemplary embodiment provides fluid compositions that are delivered intranasally and do not contain scented substances or flavoring agents, for example, odor-masking agents. Other aspects of the disclosed compositions abate respiratory odors including halitosis by clearing the nasal passages of substances that contribute to respiratory odors rather than simply masking the odors. Other aspects of the disclosed compositions abate respiratory, nasal, and/or sinus odors by reducing or eliminating the microbes (e.g., bacteria, fungi, viruses, and/or combinations thereof) that cause the odor in the first place.

The disclosed compositions are formulated not to trigger non-allergic rhinitis, and therefore can be used by individuals with sensitive nasal surfaces including individuals having vasomotor rhinitis. It has been discovered that compositions having an active ingredient including a quaternary ammonium salt are effective in reducing, controlling, abating, or eliminating odors or odor-causing agents from the nasal passages without triggering or inducing symptoms of or relating to vasomotor rhinitis.

Vasomotor rhinitis is characterized as a non-allergic, non-inflammatory nasal hyper-reactivity triggered by, for example, odors, irritants, and strong scents. The symptoms are typically wet rhinorrhea, intermittent sneezing, and stuffiness and are relative to the intensity and duration of exposure to the offending agent, as well as the individual's sensitivity to the scent or irritant. Embodiment of the compositions can be formulated to avoid triggering a symptom of vasomotor rhinitis, for example by not containing an odor-masking agent such as a scented aromatic masking agent, making the compositions suitable for use by mammals (e.g., humans) having vasomotor rhinitis. Alternatively, formulations of the compositions include ingredients that do not trigger a symptom of vasomotor rhinitis in mammals (e.g., humans) or contain a local desensitizing agent to help prevent triggering vasomotor rhinitis. Preferably the active ingredient or ingredients of the disclosed compositions are classified by the United States Food and Drug Administration as over-the-counter substances.

Another embodiment provides compositions having only one active agent, where the active agent is a quaternary ammonium salt in an amount effective to reduce odors without triggering non-allergic rhinitis when delivered intranasally, in combination with a pharmaceutically acceptable carrier suitable for intranasal delivery. Suitable quaternary ammonium salts have the following formula wherein N has a valency of 5; R¹, R², R³, R⁴ are the same or 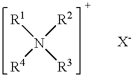
different and are independently chosen from H, an alkyl group, an alkoxy group, a cycloalkyl group, a heterocycloalkyl group, an aryl group, a heteroaryl group, an acyl group, or a thioacyl group; and X is an anion, preferably a halogen.

As used herein, the term “alkyl group” is intended to mean a straight- or branched-chain monovalent radical of saturated and/or unsaturated carbon atoms and hydrogen atoms, such as methyl (Me), ethyl (Et), propyl, isopropyl, butyl, isobutyl, t-butyl, ethenyl, pentenyl, butenyl, propenyl, ethynyl, butynyl, propynyl, pentynyl, hexynyl, and the like, which may be unsubstituted (i.e., containing only carbon and hydrogen) or substituted by one or more suitable substituents (e.g., one or more halogens, such as F, Cl, Br, or I, with F and Cl being preferred). A “lower alkyl group” is intended to mean an alkyl group having from 1 to 4 carbon atoms in its chain. Preferred alkyl groups are C₁-C₁₈, more preferably C₈-C₁₀.

An “alkoxy group” is intended to mean the radical —ORₐ, where Rₐ is an alkyl group. Exemplary alkoxy groups include methoxy, ethoxy, propoxy, and the like.

A “cycloalkyl group” is intended to mean a non-aromatic monovalent monocyclic, bicyclic, or tricyclic radical containing 3, 4, 5, 6, 7, 8, 9, 10, 11, 12, 13, or 14 carbon ring atoms, each of which may be saturated or unsaturated, and each of which may be unsubstituted or substituted by one or more suitable substituents as defined below, and to which may be fused to one or more heterocycloalkyl groups, aryl groups, or heteroaryl groups, which themselves may be unsubstituted or substituted by one or more substituents.

A “heterocycloalkyl group” is intended to mean a non-aromatic monovalent monocyclic, bicyclic, or tricyclic radical, which is saturated or unsaturated, containing 3, 4, 5, 6, 7, 8, 9, 10, 11, 12, 13, 14, 15, 16, 17, or 18 ring atoms, which includes 1, 2, 3, 4, or 5 heteroatoms such as nitrogen, oxygen, and/or sulfur, where the radical is unsubstituted or substituted by one or more suitable substituents as defined below, and to which may be fused one or more cycloalkyl groups, aryl groups, or heteroaryl groups, which themselves may be unsubstituted or substituted by one or more suitable substituents.

An “aryl group” is intended to mean an aromatic monovalent monocyclic, bicyclic, or tricyclic radical containing 6, 10, 14, or 18 carbon ring atoms, which may be unsubstituted or substituted by one or more suitable substituents as defined below, and to which may be fused one or more cycloalkyl groups, heterocycloalkyl groups, or heteroaryl groups, which themselves may be unsubstituted or substituted by one or more suitable substituents.

A “heteroaryl group” is intended to mean an aromatic monovalent monocyclic, bicyclic, or tricyclic radical containing 4, 5, 6, 7, 8, 9, 10, 11, 12, 13, 14, 15, 16, 17, or 18 ring atoms, including 1, 2, 3, 4, or 5 heteroatoms such as nitrogen, oxygen, and/or sulfur, which may be unsubstituted or substituted by one or more suitable substituents as defined below, and to which may be fused one or more cycloalkyl groups, heterocycloalkyl groups, or aryl groups, which themselves may be unsubstituted or substituted by one or more suitable substituents.

An “acyl group” is intended to mean a —C(O)—R radical, where R is a substituent.

A “thioacyl group” is intended to mean a —C(S)—R radical, where R is a substituent.

Representative quaternary ammonium compounds can be divided into the following general categories:

(1) monoalkyltrimethyl ammonium salts such as cetyltrimethylammonium bromide (CTAB);

(2) monoalkyldimethylbenzyl ammonium salts such as benzalkonium chloride;

(3) dialkyldimethyl ammonium salts; and

(4) heterocyclic ammonium salts, for example when R¹ is an alkyl chain C8-C18, remaining R², R³, and R⁴ groups are bridged to form an aromatic ring, for example pyridine in cetylpyridinium chloride. Thus, representative compounds include pyridinium quaternary salts, particularly substituted pyridinium quaternary salts such as lapirium chloride; and

(5) bisquaternary ammonium salts such as 4-aminoquinaldinium derivatives, dequalinium chloride, and hedquinium chloride.

Additional representative ammonium compounds include ipratropium bromide, hyoscine butylbromide, mepenzolate bromide, pipenzolate bromide, poldine methylsulphate, propantheline bromide, cetrimide, and domiphen bromide. The disclosed compositions can have at least one quaternary ammonium salt or combinations of multiple quaternary ammonium salts.

Flavoring agents are known in the art and can include natural or artificial flavors including natural or artificial sweeteners. The term “flavor” or “flavoring agent” as used herein refers to an agent in a form of an emulsion, concentrate, aqueous- or oil-soluble liquid or a dry powder which may be added to the compositions and does not trigger vasomotor rhinitis. Flavoring agents include, but are not limited to, any fruit flavor such as berry flavors, apple, cherry, plum, raisin, banana, pear, peach, figs, dates, lemon, coconut, kiwi, and so on. Flavoring agents may also include any nut flavors as well as any sweet flavors such as chocolate, vanilla, caramel, butterscotch, cinnamon, graham flavors, mint, and so on.

Flavoring agents additionally include any savory flavors such as all meat, game, fowl, fish, dairy, barbecue, smoke, pepper, and vegetable flavors.

The compositions also include a carrier, for example a pharmaceutically acceptable carrier. “Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as, but not limited to, phosphate, borate, citrate and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The carrier is also suitable for intranasal delivery and can include water or a mild or dilute saline solution, preferably a physiologically balanced saline solution. Additionally, the ion concentration of the carrier can be adjusted to provide a mild antibacterial effect. Saline solutions are also commonly used as moisturizers at present and are safely used in the nasal passages and sinuses. While the concentration may vary between approximately 50% and approximately 75%, a solution of approximately 65% saline has been found to be effective and non-objectionable for the user.

In still another embodiment, the control and/or elimination of objectionable odors is accomplished by clearing the nasal cavities including nasal sinuses of odor-causing agents by intranasally delivering the disclosed compositions. Odor-causing agents in the nasal cavities include microorganisms, particulates, mucous, nasal exudate, and debris. The active agent of the compositions assists in the clearing of the odor-causing agents by facilitating detachment of the odor-causing agents from nasal cavity surfaces or dissolution of the odor-causing agent. In one embodiment, the active agent facilitates the removal of odor-causing agents from nasal cavity surfaces without the need for physically forcing the odor-causing agents from the nasal cavity surfaces by forces due to irrigating the nasal cavities with the compositions. In other embodiments, the disclosed compositions solubilize or emulsify material in the nasal cavities to assist in the removal of this material from the nasal cavities.

Anti-Microbials

In yet another embodiment, the compositions contain an effective amount of an anti-microbial, for example alcohol, to provide an antibacterial effect. The alcohol is not necessary, but may facilitate mixing of the other components. Additionally, preservatives, mucolytic agents, anti-inflammatory agents, anti-histamines, desensitizing agents, or combinations thereof may be added to the compositions as needed.

Another embodiment provides compositions and methods optionally including an anti-microbial agent such as an antibiotic. Suitable antibiotics include beta-lactams such as natural and artificial penicillins and cephalosporins. Representative beta-lactams include, but are not limited to, penicillin G, and cephalothin. Semisynthetic penicillins include, but are not limited to, ampicillin, amoxycillin, and methicillin. Clavulanic acid can also be used either alone or in combination with another antibiotic such as amoxycillin sold under the tradename Augmentin®.

Monobactams such as aztreonam can also be used with the disclosed compositions. Carboxypenems such as imipenem are also useful. The class of antibiotics known as aminoglycosides including streptomycin, gentamicin, kanamycin, and tobramycin are additional representative antibiotics.

Glycopeptides such as vancomycin, lincomycins such as clindamycin and macroclides such as erythromycin and oleandomycin can also be used with the disclosed compositions. Polypeptides including polymyxin and bacitracin, rifamycins, tetracyclines such as chlortetracycline and semisynthetic tetracycline such as doxycycline can also be used. Additional antibiotics include chloramphenicol, quinolones including nalidixic acid, sulfonamides such as gantrisin and trimethoprim. Finally, isoniazid (INH), paraaminosalicylic acid (PAS), and ethambutol can be used as anti-microbials in the present invention.

Various embodiments of the composition exist in which various antiseptic and/or anti-microbial agents are used. In one embodiment of the composition, the antiseptic agent used is cetylpyridinium chloride (CPC). Other antiseptic and/or anti-microbial agents include, but are not limited to, chlorhexidine digluconate, hexetidine, sanguinanine, triclosan, and benzalkoniun chloride. Still other antiseptic agents include ethanol (1-70%) , isopropanol (1-70%), tincture of iodine (2% I₂ in 70% alcohol), silver ions such as silver nitrate (AgNO₃), and mercuric chloride. It will be appreciated that one or more of these and other known antiseptics can be include in the disclosed compositions in an anti-microbially effective amount.

Desensitizing Agents

Another embodiment provides compositions having a desensitizing agent. The desensitizing agent is an agent that assists in preventing the triggering of non-allergic rhinitis due to intranasal delivery of the disclosed compositions. Representative desensitizing agents include local anesthetics or analgesics such as antipyrine, aspirin, benzocaine, benzyl alcohol, butamben picrate, dibucaine, dimethisoquine hydrochloride, dyclonine hydrochloride, lidocaine, methyl salicylate, phenacaine hydrochloride, phenolate sodium, pramoxine hydrochloride, pyrilamine maleate, resorcinol, salicyl alcohol, salicylamide, tetracaine, thymol, tripelenamine hydrochloride, trolamine salicylate, or combinations thereof.

Mucolytic Agents

The disclosed compositions can also include a mucolytic agent to assist in the breakup of mucous. Representative mucolytic agents include ammonium chloride, antimony potassium tartrate, benzoin tincture, calcium iodide, chloroform, guaifenesin, horehound, hydriodic syrup, iodized lime, ipecac, potassium guaiacolsulfonate, potassium iodide, sodium citrate, squill, terpin hydrate, tolu, and combinations thereof.

Surfactants

As noted, the disclosed compositions optionally include a surfactant. Preferred surfactants include anionic surfactants, cationic surfactants, non-ionic surfactants, zwitterionic surfactants, and mixtures thereof. Favorable surfactants include vitamin E polyethylene glycol 1000 succinate, polyoxyethylene sorbitan fatty acid esters, polyoxyethylene stearates, polyoxyethylene alkyl ethers, polyoxyethylene castor oils, polyglycolyzed glycerides, transesterified and (poly)ethoxylated oils, sorbitan fatty acid esters, poloxamers, fatty acids salts, bile salts, alkylsulfates, lecithins, mixed micelles of bile salts and lecithins, sugar esters, and mixtures thereof. Exemplary surfactants include sodium lauryl sulfate, sorbitan monolaurate, sorbitan monostearate, polyoxyethylene sorbitan monooleate, Polyoxyl 40 Stearate, Polyoxy ethylene 50 Stearate, and bile salts.

Antirhinoviral Agents

It is known in the art that zinc ions are powerful and natural antirhinoviral agents, immune system aids, interferon inducers, cell plasma/membrane pore closing agents, anti-inflammatory agents, antioxidants, protease inhibitors, and strong drying agents. It has been found that zinc ion availability (ZIA) values of approximately 100 will shorten the common cold by an average of seven days. Prior to the composition, ZIA 100 was only available in the form of zinc acetate lozenges. The composition can also incorporate the ZIA 100 zinc acetate, or zinc chloride, or other zinc ions, thereby providing further healing and soothing properties to the composition. The zinc acetate or zinc chloride provided in the composition is pleasant tasting, flavor stable and causes no objectionable after taste.

Delivery means of the composition include for example, but are not limited to, spray bottles, droppers, and/or atomizer-type squeeze or pump bottles, or other suitable intranasal delivery systems. A one ounce (1 oz.) pump atomizer spray bottle can be an effective delivery means of the composition. Containers of this type are currently used by several pharmaceutical companies for introducing sinus medication into the nostrils and sinus cavities. The atomizer-type bottles reduce the composition to a very fine mist, thus making the delivery of the composition minimally intrusive.

No discomfort for the user was experienced in testing of the composition. In addition, no harmful effects on the nostrils or sinus cavities were caused by any of the components of the composition. Further illustrated by clinicals entitled: “A Phase Study to Evaluate the Safety and Efficacy of SinoFresh Nasal Care Used Twice Daily” (the Study). The composition effectively eliminated or masked odors associated with smoking and with eating various foods such as onion, garlic and other spices, as well as certain liquids, particularly alcoholic beverages, coffee, tea, etc.

Other than being the location of the olfactory membrane, the nasal cavity is quite unrelated to smells and smelling, but instead acts as an air-conditioning type system to clean and treat the air breathed before the air meets the sensitive and delicate tissues of the lungs. As such, the sense of smell can be improved by the cleansing of the olfactory hairs and the replacement of olfactory mucosa while using the composition of the present invention. Also, nose hairs become burdened with nicotine, tar and other tobacco-related chemicals. Routine hygienic cleansing of the nasal passages and sinus cavities aids the body in ridding itself of offensive odors, especially those that have become trapped in the middle, inferior, and superior nasal concha. Freshening ingredients of the composition that are left in the nose after cleansing with the composition leaves the nose and sinuses feeling cleansed, cleared, and refreshed with a pleasant taste.

The compositions can be used in lieu of medicated sprays, thereby relieving dependency upon medicated sprays. Without intending to be being bound by theory, it is believed that a user develops a habit with regard to the sprayer itself. For example, some reformed smokers utilize a therapeutic device resembling a cigarette to imitate the act of placing an actual cigarette in their mouth. The use of the present compositions can fulfill a similar function for the user as use of stronger, harsher medicated sprays. Second, by keeping nasal passages and sinuses cleaner and therapeutically refreshed, users can experience increased breathing capacity. This is especially beneficial because it is accomplished without the use of medicated ingredients. Therefore, the composition can be used as often as desired with absolutely no addictive problems.

Prolonged use of the compositions allows for residual deposits of the cleansing and freshening agents to pass through the nasal and sinus passage and drain down into the described area of the mouth, thus resulting in correlative improvements in reducing these bacteria and deposits. The compositions offer a more user-friendly approach to this type of bad breath.

While using the composition, individuals living in harsh, cold climates that experience cracking and bleeding of the nasal passages reported a heretofore unprecedented tremendous improvement in the cracking and bleeding symptoms, as well as improvements in their sleep distress. These improvements are a result of moisturizing agents and isotonic saline nature of the composition ingredients.

With respect to those individuals living in areas with a high pollen count, routine hygienic cleansing of the nasal passages and sinuses with the composition, can loosen and expel much of the pollen that has become trapped in the nasal passages and nose hairs which are acting as the body's filtering system for the respiratory system. The more the composition is used, the more pollen that is expelled. Because there are no medicinal ingredients in the composition, the user can cleanse the nose as often as necessary in order to relieve this type of problem.

For individuals living in polluted geographic areas, routine use of the composition can provide a previously unknown innocuous method of expelling much airborne pollution from the nose and sinuses. Use of the composition results in a cleansing and freshening process that improves breathing ability.

The compositions are also useful with resolving problems associated with the common cold. While the composition is not a cure for the common cold, many of the symptoms can be relieved without the need for medical or pharmaceutical ingredients. While the cold is “running its course”, keeping the nose and sinuses clean, bathed, and refreshed helps the individual feel and breathe much better. Engaging in a thorough hygienic cleansing just prior to retiring for bed and after arising, thereby expelling excess nasal and sinus mucus that cause a “runny nose”, brings tremendous relief to the user. Further, as noted previously, by raising the ZIA values in individuals suffering from the outset of a cold, the composition can shorten the duration of the cold.

Further, the ingredients contained in the composition are completely innocuous, i.e., there is nothing present that will interfere with the physician's care, and are believed to be readily obtainable.

With regard to individuals whose occupations require extensive verbal communication, unexpected results indicate that users of the composition have been able to restore moistness to their noses and sinuses and during routine use, residual amounts of the ingredients pass through the nose and sinuses, leaving deposits of the cleansing, freshening agents to soothe the throat.

The routine, hygienic cleansing of the nose and sinuses, using the composition containing isotonic saline, moisturizing, purifying, and antiseptic agents will result in cleaner, healthier nasal and sinus passages, rewarding the user with an improved breathing ability. Users of the composition can obtain relief of many types of symptoms, likely due to their routine programs of hygienic cleansing of the nose and sinuses.

### EXAMPLE 1

| Ingredient | Amount |
|---|---|
| Sodium chloride | About 0.65% |
| Methyl salicylate | About 0.02% |
| Benzalkonium chloride | About 0.002%-0.015% |
| PEG or glycerin | Trace (Optional) |
| Zinc acetate/zinc chloride | Optional |
| Thimerosal | About 0.001% |

The above listed formula for the composition has undergone testing and has proved effective for its stated purpose. The composition was dispensed from a 1 oz. pump atomizer bottle and was successful in eliminating and/or masking odors which could be considered offensive in certain circumstances or by certain people, i.e., odors from smoking, cutting, dicing, or consuming onions, etc.

Based on experiments that have been conducted, the methyl salicylate and menthol can be replaced by other similar acting ingredients to completely change the flavor. The base ingredients of the composition, preservative(s) and alcohol, are in percentage amounts that will remain relatively constant. The solution of the invention is prepared according to known techniques and excipients, as described in “Remington's Pharmaceutical Sciences Handbook,” 17^{th} ed., Hack Publ. Co., N.Y., U.S.A.

It should be stated that the composition of the invention is not limited to the ingredients stated in the original application. Substitution ingredients are widely available for each of the essential components, as described in “Remington's Pharmaceutical Sciences,” 17^{th} edition.

The first set of intrinsic ingredients of the composition is the antiseptic and anti-infective agents. The elimination of certain types of bacteria, which are known to cause offensive odors, will aid in the freshening of the breath and are chosen and formulated to produce the results as described.

The second intrinsic ingredient of the composition is the isotonic, aqueous solution, acting as an osmotic agent. This ingredient is chosen and formulated for the isotonic value and fully disclosed in “Remington's Pharmaceutical Sciences,” 17^{th} edition. This ingredient is chosen and formulated to produce the results further described herein.

### EXAMPLE 2

The following formula provides a second representative example of an isotonic, sterile, aqueous solution of the composition. In this formula, the function of the ingredients is given under “Application”.

| Range | Agent | Description | Percentage (W/W) |
|---|---|---|---|
| About 0.1 to 2% | Sodium chloride | Osmotic agent | About 0.650 |
| About 0.1 to 5% | Sodium borate | Buffering agent | About 0.100 |
| About 0.1 to 9% | Alcohol SD | Solubilizing agent | About 0.090 |
| About 0.001 to 2% | Edetate disodium | Preservative | About 0.050 |
| About 0.1 to 3% | Glycerin | Solubilizing agent | About 0.001 |
| About 0.001 to 5% | Poloxamer 407 | Antiseptic | About 0.030 |
| About 0.001 to 5% | Domiphen bromide | Antiseptic | About 0.030 |
| About 0.001 to 5% | Cetylpyridinium chloride | Anti-infective | About 0.040 |
| About 0.1 to 2% | Sorbitol | Sweetener | About 0.002 |
| About 0.1 to 2% | Sodium saccharin | Sweetener | About 0.002 |
| About 0.1 to 5% | anesthetic | desensitizing agent | About 0.005 |
| About 0.001 to 5% | Zinc acetate/zinc chloride | Healing agent | About 0.040 |
| | Deionized water | Solvent (vehicle) | About <100% |
| About 0.001 to 5% | Polysorbate 80 | Surfactant | About 0.045 |

It should be emphasized that the above-described embodiments, particularly any “preferred” embodiments, are merely possible examples of implementations, and are merely set forth for a clear understanding of the principles set forth herein. Many variations and modifications may be made to the above-described embodiment(s) of the compositions and methods without departing substantially from the spirit and principles of the disclosure. All such modifications and variations are intended to be included herein within the scope of this disclosure and protected by the following claims.

1. A composition for cleaning nasal cavities comprising:
an active agent consisting essentially of a quaternary ammonium salt and a pharmaceutically acceptable carrier suitable for intranasal delivery, wherein the composition is formulated to reduce odors without triggering non-allergic rhinitis when delivered intranasally.

2. The composition according to claim 1, wherein the quaternary ammonium salt is chosen from the at least one quaternary ammonium salt of the following formula: 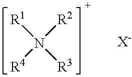
wherein N has a valency of 5;
R¹, R², R³, R⁴ are the same or different and are independently chosen from H, an alkyl group, an alkoxy group, a cycloalkyl group, a heterocycloalkyl group, an aryl group, a heteroaryl group, an acyl group, or a thioacyl group; and
X is an anion, preferably a halogen:

3. The composition according to claim 2, wherein two or more of R¹, R², R³, and R⁴ share at least one chemical bond.

4. The composition according to claim 2, further comprising a desensitizing agent.

5. The composition according to claim 2, further comprising at least one of a mucolytic agent, an antibiotic, a flavoring agent, and combinations thereof.

6. The pharmaceutical composition according to claim 2, wherein said carrier includes a component chosen from a buffer to maintain a physiological pH of said composition, a pharmaceutically acceptable thickening agent, a humectant, a pharmaceutically acceptable surfactant, and combinations thereof.

7. The pharmaceutical composition according to claim 2, further comprising at least one pharmaceutical excipients.

8. The pharmaceutical composition according to claim 7, further comprising a pharmaceutically acceptable preservative.

9. The pharmaceutical composition according to claim 6, wherein said buffer is chosen from at least one of acetate, citrate, borate, phosphate buffers, and combinations thereof.

10. The pharmaceutical composition according to claim 6, wherein said thickening agent is chosen from at least one of methyl cellulose, xanthan gum, carboxymethyl cellulose, hydroxypropyl cellulose, carbomer, and mixtures thereof.

11. The pharmaceutical composition according to claim 6, wherein said humectant is chosen from at least one of sorbitol, propylene glycol, glycerol, and mixtures thereof.

12. The pharmaceutical composition according to claim 6, wherein said surfactant is chosen from at least one of polyoxyethylene derivatives, fatty acid partial esters of sorbitol anhydrides, and combinations thereof.

13. The pharmaceutical composition according to claim 6, wherein said surfactant is chosen from at least one of polyoxyethylene sorbitan fatty acid esters, polyoxyethylene stearates, polyoxyethylene alkyl ethers, polyoxyethylene castor oils, polyglycolyzed glycerides, transesterified and (poly)ethoxylated oils, sorbitan fatty acid esters, poloxamers, poloxamines, fatty acids salts, bile salts, alkylsulfates, lecithins, mixed micelles of bile salts and lecithins, sugar esters, and mixtures thereof.

14. The pharmaceutical composition according to claim 6, wherein said surfactant is chosen from at least one of anionic, cationic, nonionic surfactants, and combinations thereof.

15. A method of cleaning nasal cavities in a mammal comprising nasally delivering to the mammal an active agent consisting essentially of a non-alcoholic cleaning agent and a pharmaceutically acceptable carrier suitable for intranasal delivery, wherein said composition is formulated to not trigger non-allergic rhinitis.

16. The method according to claim 15, wherein said carrier comprises a buffer to maintain the pH of said composition, a pharmaceutically acceptable thickening agent, a humectant, a flavoring agent, a pharmaceutically acceptable surfactant, and combinations thereof.

17. The method according to claim 16, further comprising at least one pharmaceutical excipients.

18. The method according to claim 17, further comprising a pharmaceutically acceptable preservative.

19. The method according to claim 16, wherein said buffer is chosen from at least one of acetate, citrate, phosphate buffers, and combinations thereof.

20. The method according to claim 16, wherein said thickening agent is chosen from at least one of methyl cellulose, xantham gum, carboxymethyl cellulose, hydroxypropyl cellulose, carbomer, and mixtures thereof.

21. The method according to claim 16, wherein said humectant is chosen from at least one of sorbitol, propylene glycol, glycerol, and mixtures thereof.

22. The method according to claim 16, wherein said surfactant is chosen from at least one of polyoxyethylene derivatives, fatty acid partial esters of sorbitol anhydrides, and combinations thereof.

23. The method according to claim 16, wherein said surfactant is chosen from at least one of polyoxyethylene sorbitan fatty acid esters, polyoxyethylene stearates, polyoxyethylene alkyl ethers, polyoxyethylene castor oils, polyglycolyzed glycerides, transesterified and (poly)ethoxylated oils, sorbitan fatty acid esters, poloxamers, poloxamines, fatty acids salts, bile salts, alkylsulfates, lecithins, mixed micelles of bile salts and lecithins, sugar esters, and mixtures thereof.

24. The method according to claim 16, wherein said surfactant is chosen from at least one of anionic, cationic, and nonionic surfactants.

25. The method of claim 15, wherein the dosage rate of the composition administered to a mammal is in an amount from 1 mg to about 10 g.

26. The method of claim 15, wherein the composition contains concentrations of a non-alcoholic cleaning agent in a range from about 0.001% to about 10% by weight.

27. The method of claim 15, wherein the administration rate comprises administering said composition at a rate of about once daily to about 4 times per day.

28. A composition for treating non-allergenic rhinitis comprising an active agent consisting essentially of a non-alcoholic cleaning agent and a pharmaceutically acceptable carrier suitable for intranasal delivery,
wherein the composition is formulated to reduce odors without any flavoring agent or scented substance, and
wherein the composition is formulated not to trigger non-allergic rhinitis when delivered intranasally.

29. A composition for treating non-allergenic rhinitis comprising an active agent, the active agent consisting of a non-alcoholic cleaning agent and a pharmaceutically acceptable carrier suitable for intranasal delivery,
wherein the composition is formulated to reduce odors without any flavoring agent or scented substance, and
wherein the composition is formulated not to trigger non-allergic rhinitis when delivered intranasally.
